(19) United States
(12) Patent Application Publication
(10) Pub. No.: US 2018/0126052 A1
(43) Pub. Date: May 10, 2018

(54) Automated Compression Nursing And Pumping System

(71) Applicant: Lilu, Inc., Wilmington, DE (US)

(72) Inventors: Alexandra Catharine Looney, Wilmington, DE (US); Sujay Suresh Kumar, Philadelphia, PA (US); Adriana Catalina Vazquez Ortiz, New York, NY (US)

(21) Appl. No.: 15/598,050

(22) Filed: May 17, 2017

Related U.S. Application Data
(63) Continuation of application No. 15/373,752, filed on Dec. 9, 2016.
(60) Provisional application No. 62/419,747, filed on Nov. 9, 2016.

(51) Int. Cl. A61M 1/06; A41C 3/04; A41C 3/10

(52) U.S. Cl. CPC A61M 1/064; A41C 3/105; A41C 3/04

(57) ABSTRACT

A system for hands-free nursing and breast pumping includes a breast compression system that uses moldable chambers filled with air inside the cups of a nursing bra or attached to the flange of a breast pump to apply pressure and massage patterns to a breast while using a breast pump. This compression or pressure is radially applied on the surface of the breast, and its rhythm and strength may be adjusted by the user through the use of a controller in order to mimic the best practices of manual compression. These bra cups with the compression device have openings over the nipples so that a breast pump flange can be inserted through them and used in a typical manner for the extraction of breast milk, thus increasing the amount of milk expressed.

### CROSS-REFERENCE TO RELATED APPLICATIONS

The present application is a Continuation of U.S. patent application Ser. No. 15/373,752 filed Dec. 9, 2016, which claims priority to U.S. Provisional Patent Application No. 62/419,747, filed Nov. 9, 2016. The contents of that application are hereby incorporated by reference.

### BACKGROUND

It is known that manual breast massage increases the amount of milk expressed in a breastfeeding session (Bowles, B. C. (2011). Breast Massage: A “Handy” Multipurpose Tool to Promote Breastfeeding Success. Clinical Lactation, 2(4), 21-24.). Health benefits for the mother of compression massage during breastfeeding or breast pumping include preventing and relieving mastitis, plugged ducts, and engorgement (Witt, A. M. et. al. (2016). Therapeutic Breast Massage in Lactation for the Management of Engorgement, Plugged Ducts, and Mastitis. Journal of Human Lactation, 32(1), 123-31.). When combined with electric pumping, manual massage both increased milk production (Morton, J. et. al. (2009). Journal of Perinatology, 29, 757-64.) and caloric content (Morton, J. et. al. (2012). Journal of Perinatology, 32, 791-96.) for preterm infants. The benefit is not limited to preterm infants, however. Compression stimuli around the areola increases breast pump efficiency more generally by stimulating the release of necessary hormones (Alekseev, N. P. et. al. (1998). Compression stimuli increase the efficacy of breast pump function. European Journal of Obstetrics & Gynecology and Reproductive Biology, 77(2), 131-39.). While manual massage has been used for centuries, the ability to pump efficiently hands-free would greatly improve the modern mother's productivity.

There are existing systems that combine hands-free compression and pumping. U.S. Pat. No. 6,213,840 B1, for example, discloses a simple hands-free bra that supports a breast pump system. It does not, however address the need for compression. Some breast pumps have been designed to mimic manual compression, such as that described by the application US 2005/0234370 A1, which discloses that pressure is applied by a “plurality of opposing pairs of expression bellows.” At least two patent applications describe hands-free compression systems to be used in conjunction with breast pumps using pneumatic modes of compression (US 2014/0378946 A1 and US 2015/0065994 A1). However, the prior art does not incorporate systems or methods that effectively mimic the best practices of manual compression, as indicated by research and expertise of lactation specialists.

### SUMMARY

The present invention addresses the needs in the prior art by providing rotating compression pressure across multiple hemispheres of the breasts in a pulsing manner, more closely mimicking manual compression. The system and methods described herein further incorporate the existing research and existing expertise of lactation specialists to provide a controllable, hands-free compression and breast pumping system.

In particular, the system described herein provides hands-free expression of milk from human breasts in a rotating manner across the hemispheres of the breast to mimic natural manual compression. The system comprises a compression device and a controller that controls the compression device to improve upon manual techniques for milk expression to allow for a hands-free breast pumping or enhanced nursing experience. The embodiments described herein incorporate a compression device, a controller and, optionally, a specially designed bra. The bra is designed to include an opening in each cup to lay over the nipple for the user to attach a suction breast pump or alternatively, the baby may latch onto the breast through the opening while the compression device is attached and operating to increase expression of milk. In such an embodiment, the compression device is incorporated into the bra, which is further designed to allow for simultaneous breast pumping or nursing. In another embodiment, the specially designed bra is not needed as the compression device is incorporated onto a breast pump, thus combining compression and suction. In this embodiment, the controller for the compression device is integrated into the controller for the breast pump.

The compression device is controlled to compress different segments of the breasts in a rotational, hemispheric manner. This approach incorporates the research and best practices of lactation, by applying pressure to the breasts in a way that mimics manual compression. The compression device comprises at least one inflatable bladder to lay over the breast, with an opening in the center of the device to lay over the nipple. The compression device is configured to apply compression to the breasts in a rotating, pulsing manner. Additionally, the compression device is pneumatically actuated through tubes that pass from the controller into the compression device. The controller increases or decreases the pressure exerted by the compression device by increasing or decreasing the fluid pumped.

In a first embodiment, the compression device comprises a number of inflatable arms arranged radially around the nipple. These arms may vary in length to reach the tail of Spence, the outer diagonal hemisphere of the breast, or the inner hemispheres of the breasts. The arms may each have their own tube connecting them to the controller, or they may share air tubes. The arms inflate and deflate in rotating hemispheric massage patterns. In this embodiment, the compression device is embedded into a nursing bra.

In a second embodiment, the compression device includes inflatable bladders that are disc-shaped with an opening in the center, and inflation proceeds in a rotational manner. The inflatable bladders have one or more sealed chambers, each designed such that air passes from the air tube into a larger pocket, proceeding through smaller channels into other larger pockets, inflating the chambers rotationally. Each chamber may include its own air tube, or the chambers may share an air tube. The inflatable bladders may be inserted into the bra through a sleeve. At least one small rigid protrusion may be attached to the compression device and facing the user's breast for additional pressure when the bladder is inflated. Further, a semi-rigid element serves to prevent the inflatable bladders from inflating away from the breasts, forcing the pressure to compress the user's breasts.

In a third embodiment, the compression device of either the first or second embodiment is attached to a suction breast pump instead of being incorporated into a nursing bra. Instead of an opening that lays over the nipple, the center of the compression device comprises a flange for a suction breast pump. The controller for the compression device and the controller for the vacuum-based suction breast pump are housed in the same unit.

### BRIEF DESCRIPTION OF THE DRAWINGS

The present invention will be more fully understood from the detailed description below taken in conjunction with the accompanying drawings, in which:

FIGS. 1 through 5 disclose a first embodiment of the system. FIGS. 6 through 11 disclose a second embodiment of the system. FIG. 12 discloses the components of the controller in the second embodiment. FIGS. 13 through 17 disclose a third embodiment of the system.

FIG. 1A is a perspective anterior view of a bra in which the compression device may be integrated;

FIG. 1B is a perspective posterior view of a bra in which the compression device may be integrated;

FIG. 2 is a perspective view of the front of the compression device;

FIG. 3 is a perspective view of the back of the compression device;

FIG. 4 is a drawing of a massage actuator, the petals of which are pneumatically inflated and deflated with fluid to massage the breast;

FIG. 5 is a perspective view of a controller in which the user can control the strength of the compression and where the massage actuator inflates;

FIG. 6 is a perspective view of a bra having first and second sleeves in which the compression device may be inserted, as well as openings that fall over the user's nipples such that an external breast pump may be attached;

FIG. 7 is a cross-section perspective demonstrating how the bra and compression device lay over a user's breast;

FIG. 8 is a perspective view demonstrating how the compression device is to be inserted into the bra;

FIG. 9A is a first design of the compression device to be integrated into the bra, exhibiting a massage actuator, the chambers of which inflate to massage the breast;

FIG. 9B is a second design of the compression device to be integrated into the bra, exhibiting a massage actuator, the chambers of which inflate to massage the breast with non-flexible protrusions to provide additional pressure;

FIG. 9C is a third design of the compression device to be integrated into the bra, exhibiting a massage actuator, the chambers of which inflate to massage the breast with multiple non-flexible protrusions to provide additional pressure;

FIG. 10 is an exploded view of the compression device, demonstrating three layers: the first layer consisting of rigid protrusions that lay on the breast, the second layer of inflatable elements, and the third layer of a semi-rigid backing;

FIG. 11 is a perspective view of a controller for the user to control the pressure and rhythm of the massage actuator;

FIG. 12 is a high-level block diagram of the components operating the compression device;

FIG. 13 is a perspective view of the system comprising the compression device, pump, and controller for the compression device and pump;

FIG. 14 is a perspective view of the compression device facing the user, with the compression device fitted onto a breast pump flange;

FIG. 15 is a perspective view of the compression device as it would lay on a user's breast;

FIG. 16 is a perspective view of a controller in which the user can control the strength of the compression, the strength of suction, and the pattern of compression;

FIG. 17 is a high-level chart that displays the components of the controller and how the components work with each part of the system; and

FIG. 18 is a similar high-level chart that displays the components of the controller and how the components work with each part of the system, with one air and vacuum pump instead of two.

### DETAILED DESCRIPTION OF EXEMPLARY EMBODIMENTS

Certain embodiments will now be described to provide an overall understanding of the principles of the structure, function, manufacture, and use of the devices and methods herein disclosed. One or more examples of these embodiments are illustrated in the accompanying drawings, briefly described above.

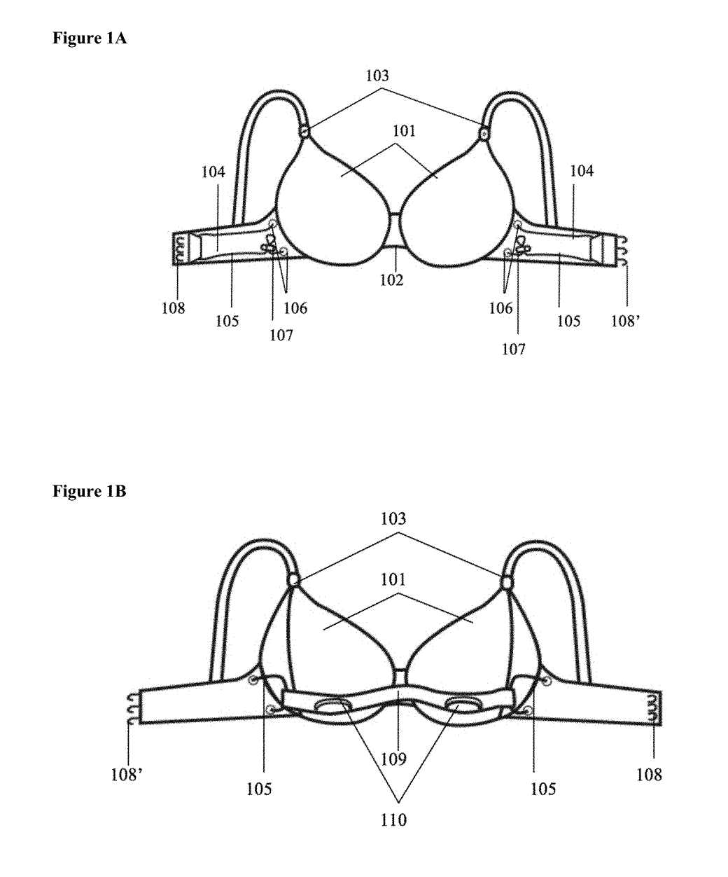

### Embodiment 1—FIG. 1A Through FIG. 5

FIG. 1A and FIG. 1B are perspective views of a bra that has been configured to accommodate both a compression device as well as a breast pump. The bra includes two breast cups (101) connected by a center panel (102). Each breast cup (101) can be individually unclipped from the bra strap (103) to allow for access to the breast and nipple area. The bra wings (104) have an elastic cord (105) which threads through the bra wings (104) through a reinforced slit (106) and connects the bra wings (104) to a band (109) on the inside of the bra. An adjustable toggle (107) is attached to a loop in the elastic cord (105) so that it can be pulled to adjust the tightness of the inner band (109) and the bra wing (104) while the user is still wearing the bra. The bra can be connected and disconnected using hooks and loops (108, 108′) attached to the end of each bra wing (104). Bordering the lowest edge of the bra where the base of the breasts would reside is an inner band (109) used for attaching the compression device into the nursing bra via loops (110) through which the bottom of the compression device can be inserted. The outer elastic cord (105) can be threaded through the inner band (109) and tightened by using the adjustable toggle (107) for a better fit.

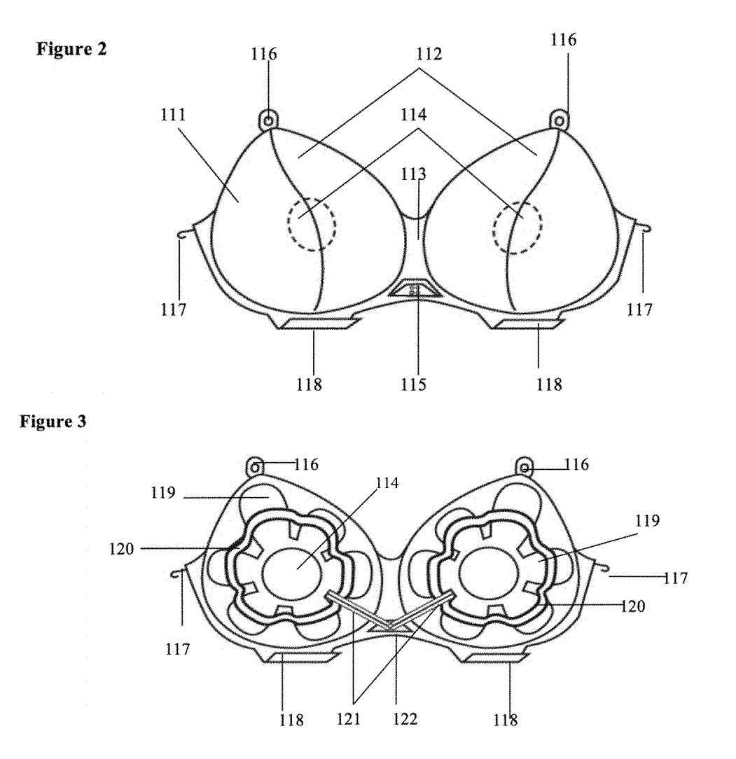

FIG. 2 illustrates the outside of the compression device (111), which includes two bra cups (112), separated by a center panel (113). Each cup (112) has an opening (114) that lays over the nipple to allow a breast pump flange to reach the nipple. In the center of the center panel is a port (115) in which air tubes can be connected and/or disconnected to allow for airflow to pass from the controller to the inner air chambers of the massage actuator that are on the inside of the breast cups of the compression device. This port (115) allows for these tubes to be connected and secured to provide an airtight connection, but which can easily be disconnected by the user. It is required for this port (115) to be connected to the main controller via tubes for the massage actuator to function. The compression device is attached to the nursing bra in multiple places for stability and comfort (116, 117, 118).

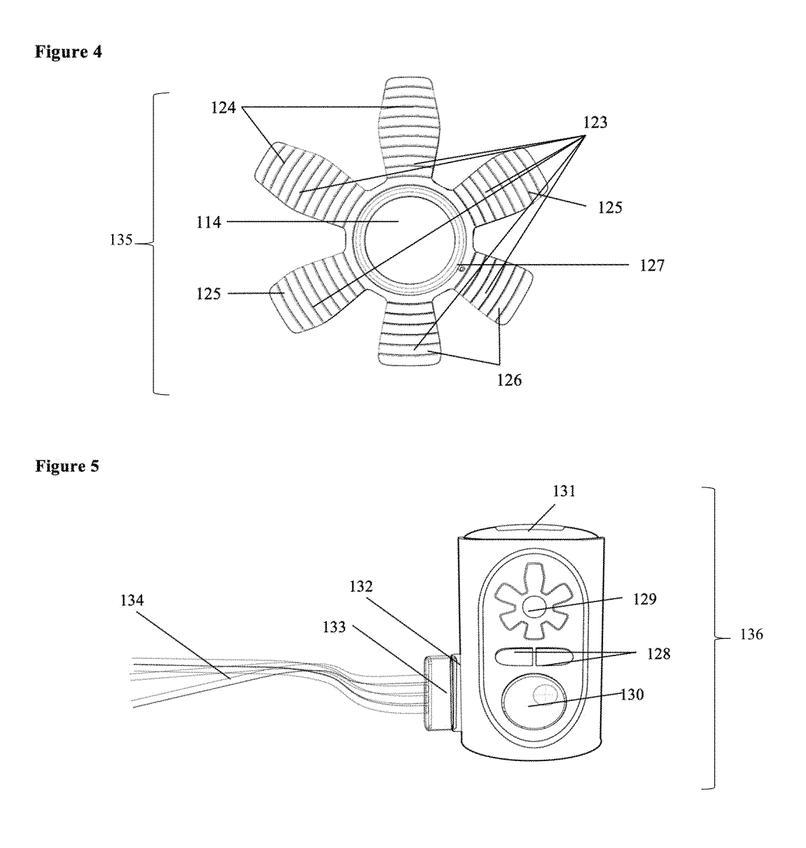

FIG. 3 illustrates the inside of the compression device (111), which includes a convex form (119) to hold the massage actuator (135). As shown in FIG. 4, the massage actuator (135) is formed in a radial design so as to apply pressure to the surface of the breast starting on the furthest end of each arm (123) and moving the pressure down the length of the arm toward the nipple of each breast. Each arm (123) of the massage actuator (135) is attached to the inside of the bra cup (112) with firmly attached straps (120) so that it is free to move when air flow has been pushed through the air tubes (121) but is firmly in place so that it does not rotate around the nipple. The massage actuator (135) is powered by air tubes (121) which come through the center panel (122) of the compression device. These tubes make an airtight connection with the massage actuator (135) and therefore allow air to flow through the air tubes (121) and into the massage actuator (135). This flow of air causes the arms (123) of the massage actuator (135) to curl thereby applying pressure onto the surface of the breast.

FIG. 4 is a massage actuator (135) that may be constructed from a variety of materials (including but not limited to rubber, silicone, plastic, etc.), including a number of inflatable arms (123) of equal or varying length. Each arm (123) has been designed to massage the most alveolar glands as well as milk ducts in that portion of the breast. The arms (123) can cover the entire surface area breast, and may include the following: two long arms (124) that may reach and massage the tail of Spence; two intermediate arms (125) that may reach and massage the outer diagonal hemispheres of the breast; and two small arms (126) which may reach and massage the inner hemispheres of the breast. The air channels (127) connecting the arms may all be activated at once. Air channels (127) may be separated to allow rotating hemispheric massage patterns that may promote milk extraction. The opening in the center of FIG. 4 (114) should match up with the openings in FIG. 2 and FIG. 3. As shown in FIG. 3, the massage actuator (135) is inflated by air through one or more tubes (121) that connect the compression device to the controller (136) shown in FIG. 5. Air tubes (121) connect from the controller (136) to the arms (123) of the massage actuator (135). The arms (123) may be connected by, and share one or more air tubes (121), or each arm (123) may connect individually to the controller (136) with its own tube (121).

FIG. 5 illustrates a controller (136) for controlling the compression device. The user can control the pressure and rhythm of the massager by manipulating the pressure and rhythm button (130). The next adjustment function is provided by the left and right power buttons (128). The user can also turn the air on or off on each individual massager thereby deciding if they want pressure applied on their right or left breast or both at the same time. The next adjustment feature is provided by the pattern adjustment button (129). This allows the user to modulate the motion pattern of the arms of the massager. The arms (123) of the massage actuator (135) can be engaged and apply pressure all at once. This causes all the arms (123) to curl at the same time. The user may also use this button to cause a portion of the arms which are symmetrical to each other (124, 125, 126) across the nipple to curl and engage while the other arms remain still. After the first set of arms engage, the remaining set of arms engage and apply pressure while the first set of arms remain still. The frequency and strength of the compression pattern of rotating pressure from the arms is adjusted by the pressure and rhythm button (130). The final adjustment feature is the on and off switch (131), which controls if the controller (136) is powered or not. The airflow is routed out of the controller (136) by an air tube port (132). This port allows for the air tubes (134) that connect to the compression device to be easily snapped into place with the use of a port connector (133) that is attached to the ends of the air tubes (134). This connection when placed inside the controller's air tube port (133) allows for an airtight seal which maintains the PSI generated by the controller (136) and allows it to flow while maintaining pressure to the massage actuator (135) within the compression device. An algorithm programs the controller to respond to the respective settings to selectively inflate and deflate the arms of the massage actuator to mimic manual compression of the breasts with the desired pattern and frequency.

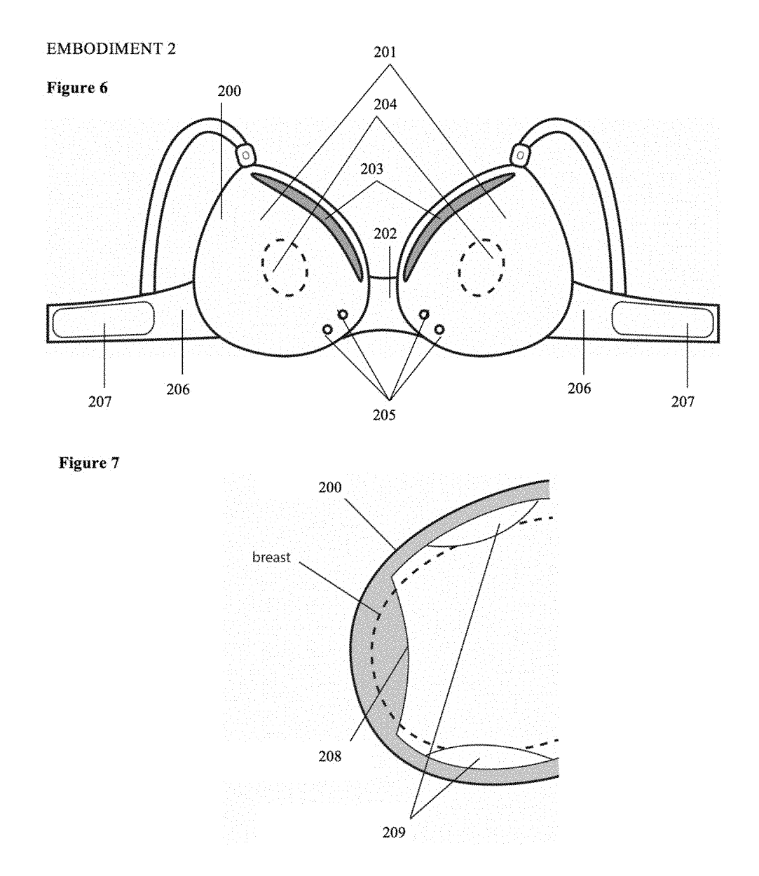

### Embodiment 2—FIG. 6 Through FIG. 11

FIG. 6 is a perspective view of a bra (200) that has been configured to accommodate both the compression device (208) as well as a breast pump. The bra (200) includes two breast cups (201) separated by a center front panel (202). Each breast cup (201) has a sleeve (203) into which the compression device (208) is inserted, as well as an opening (204) that lays over the nipple. Additionally, there are small openings (205) in each breast cup (201) into which tubes (212) can connect the controller (221) to the compression devices. The wings (206) of the bra (200) have a material (e.g., VELCRO®) (207) that allows for adjustable sizing by the user.

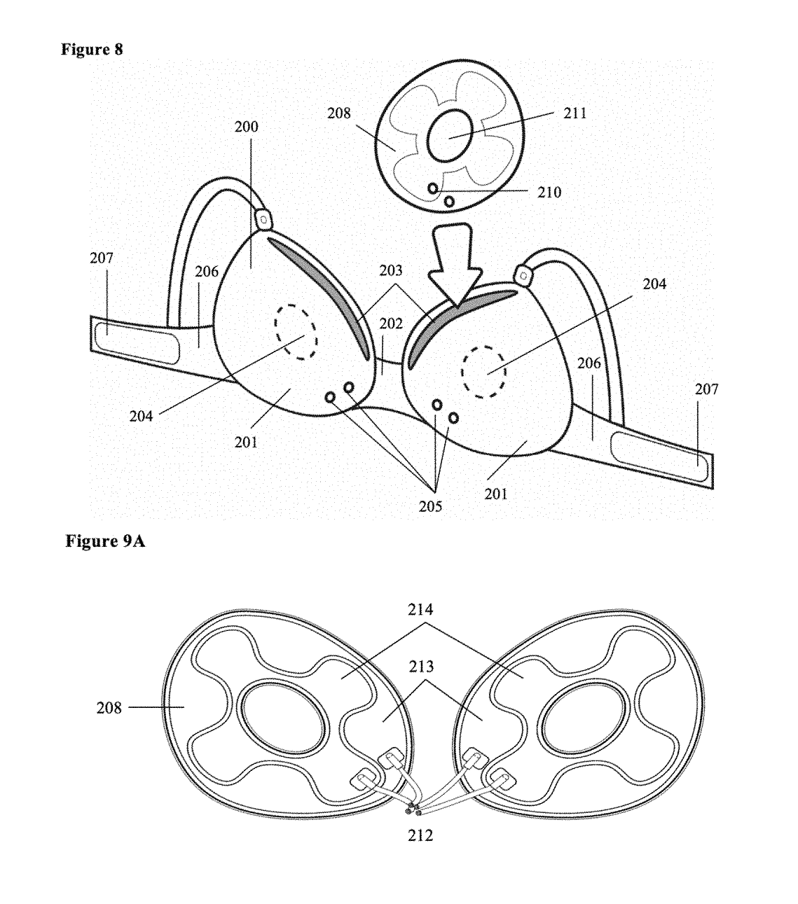

FIG. 7 demonstrates how the compression device (208) and bra (200) fit over a user's breast, indicated by the curved, dotted line. The compression device (208) is inserted into the bra (200), and lays over the breast. The compression device air channels (209) serve to put pressure onto parts of the breast, mimicking manual compression.

FIG. 8 demonstrates how the compression device (208) is to be inserted into the bra (200) through the sleeve (203) in the seam of the bra cup. The air ports (210) on the compression device (208) line up with the openings in the bra (205) such that the user can attach the air tubes (212) to the compression device through the bra (200) once it is inserted in the bra sleeves (203). The opening (211) in the compression device (208) should also line up with the opening in the bra cup (204).

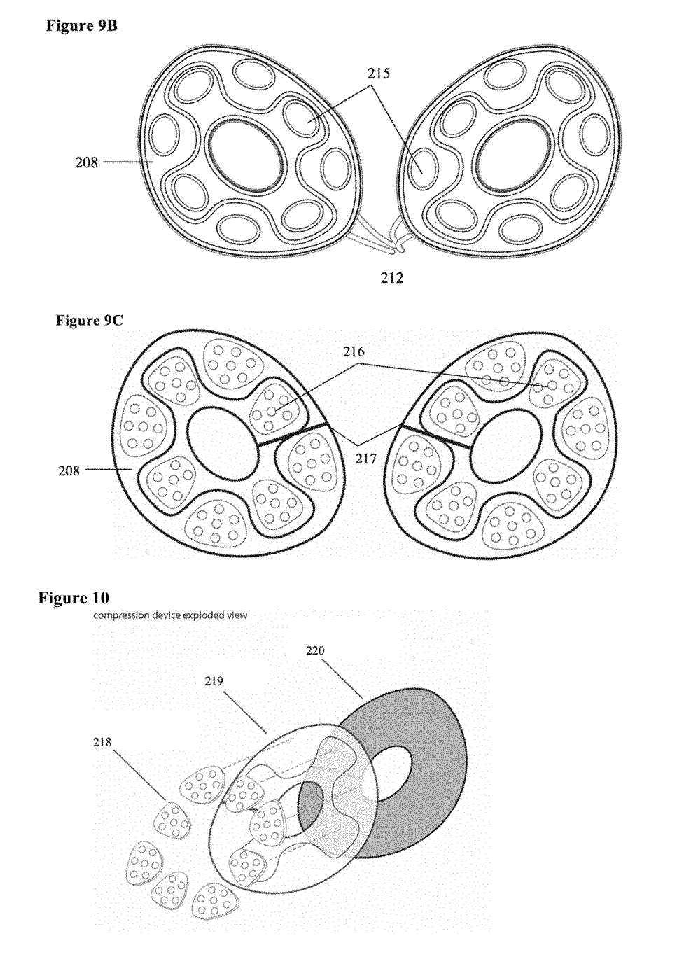

FIGS. 9A-C are variations of the compression device (208). FIG. 9A show how air tubes (212) may connect to the compression device (208). Each compression device (208) is composed of at least two inflatable chambers (213, 214), each chamber connected to its own air tube (212). The chambers (213, 214) inflate and deflate in an alternating pattern. Because of the design of the chambers, the air flows from the air tube (212) into the chamber, filling a large pocket before being pushed through a smaller channel before entering into another large pocket, and so on and so forth until the entire chamber (213) has inflated in a rotational pattern. Once one chamber (213) is fully inflated, it deflates as the other chamber (214) begins to inflate. The air passes in and out of the tubes (212) connecting the compression device to the controller (221). FIG. 9B demonstrates the compression device with rigid protrusions (215) (made from a variety of materials, including hard rubber and plastic), attached to the large pockets of each chamber (213, 214), adhered to the side facing the user to add additional pressure. FIG. 9C demonstrates that the bra cups may also have multiple rigid elements (216) adhered to the side of the bra facing the user. These elements have small protrusions (indicated by the circles) which transfer pressure during the inflation of the compression device (208). The black bar on the side of the compression device indicates a seam (217) which allows the air to flow through the air channels from the bottom of the device to the top, creating a rotating pattern of inflation.

FIG. 10 is an exploded view of the compression insertion (208), illustrating three potential layers of construction. The first layer of rigid protrusions (218) is the layer closest to the user's breast. The second layer comprises inflatable air bladders (219). The third layer, made of a semi-rigid backing (220) (made of plastic or other semi-rigid material), forces the inflation of the second layer (219), to apply pressure to the breast.

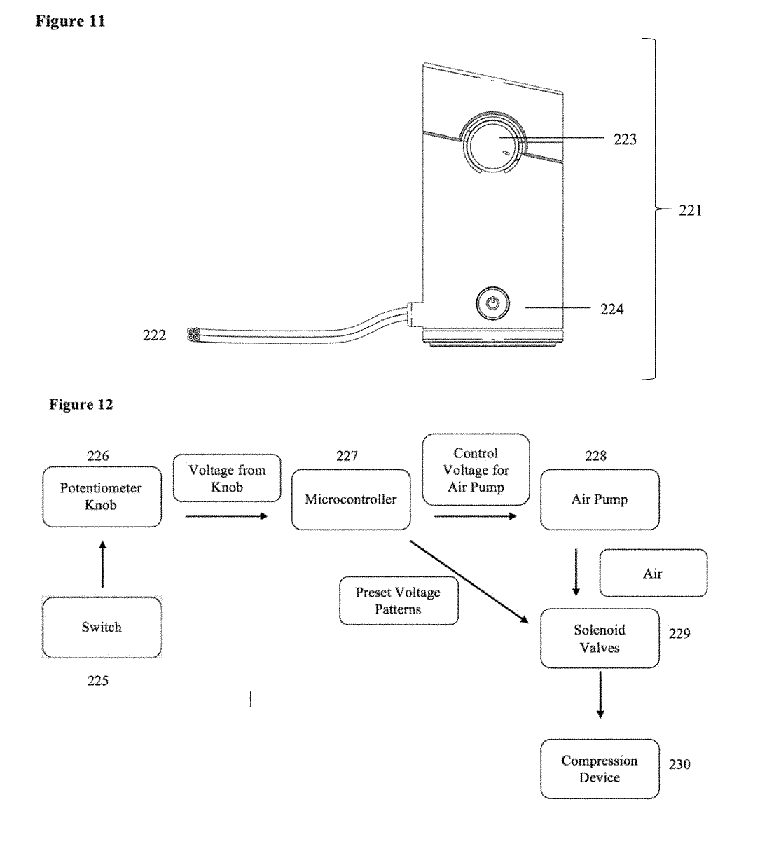

FIG. 11 illustrates the controller (221) in which the user can turn on the device by pressing a button (224) and choose the strength of the compression by turning a dial (223), which corresponds to an increase in the amount of air pumped into the compression device through the tubes (222).

FIG. 12 applies to both of the above-described embodiments as a high-level chart indicating the components of the controller and how they interact with each other. The components of the controller (FIG. 11) are the switch (225), the potentiometer knob (226), the microcontroller (227), the air pump (228), and the solenoid valves (229). The switch (225) turns the device on or off by supplying or cutting off the supply of voltage. The potentiometer knob (226) is a rotating knob (223) used to control the amount of pressure exerted by the compression device by gradually increasing the output voltage between zero and maximum voltage when turned in one direction, and gradually decreasing the output voltage between maximum and zero voltage when turned in the opposite direction. The microcontroller (227) automates the entire system; the functionalities of the switch (225), the potentiometer knob (226), the control voltage for the air pump and the control voltages for both the solenoid valves (229) are coded into the microcontroller (227). The source of compressed air is the air pump (228), which is composed of two or more nozzles. Air is sucked into the air pump from one nozzle, compressed and sent out through the other. The amount of air output by the pump can be altered by the amount of voltage given to the pump by the microcontroller (227). The solenoid valves (229) are electric valves that act like a gate. They contain multiple nozzles, some for input and some for output. The air output from the air pump is supplied to the input of the solenoid valves (229). When voltage is supplied to the valve, it lets the air out from one of the output nozzle. When voltage is turned off, the other output nozzle is open to let the excess air in the system out. By controlling the voltage to these valves, the air supplied to the compression device can be turned on and off.

The microcontroller (227) controls both the air pump (228) and the solenoid valves (229). The microcontroller (227) reads the voltage output from the potentiometer knob (226) to determine the strength of the pump (228). The microcontroller (227) then controls the pump (228) by adjusting the voltage supply. The air output from the pump is fed to the solenoid valves (229). The microcontroller (227) controls the solenoid valves by supplying voltage to it and supplies voltage to these valves in a preset pattern. The solenoid valve (229) gets the air from the pump (228). When the valves (229) are turned on by the microcontroller (227), the air is sent to the air channels in the compression device (230), using tubes (212). When the valves are on, air is supplied to the air tubes (212) and the chambers (213, 214) inflate. When the valves (229) are off, the excess air in the air channels in drained off from the solenoid valves. An algorithm programs the controller (221) to respond to the respective settings to inflate and deflate the chambers (213, 214) of the compression device (208) to mimic manual compression of the breasts having a desired pattern and frequency of breast compression.

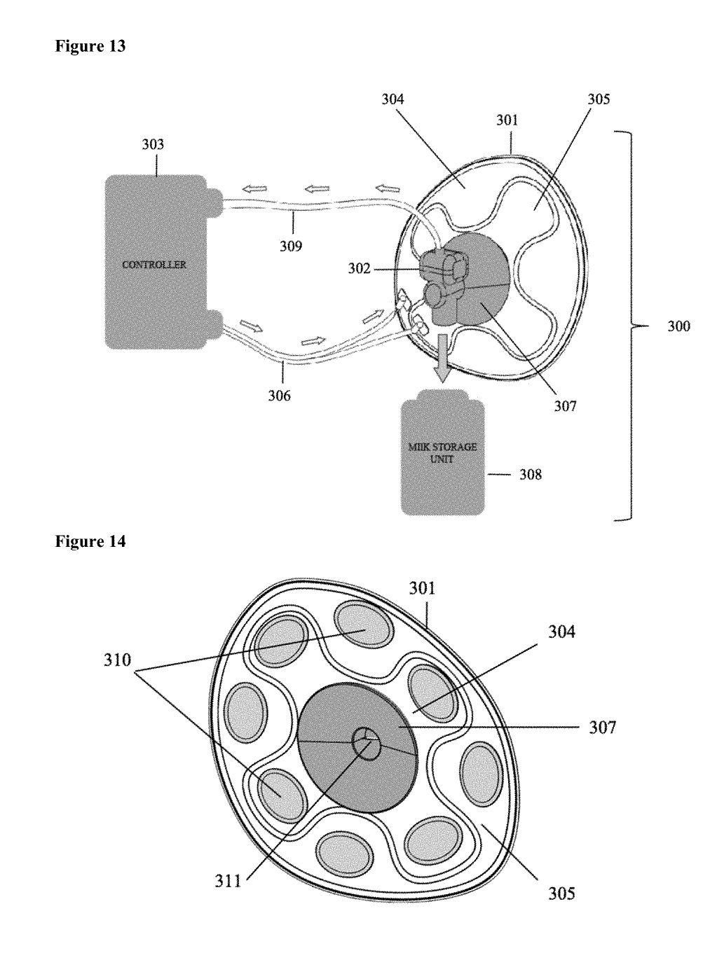

### Embodiment 3—FIG. 13 Through FIG. 17

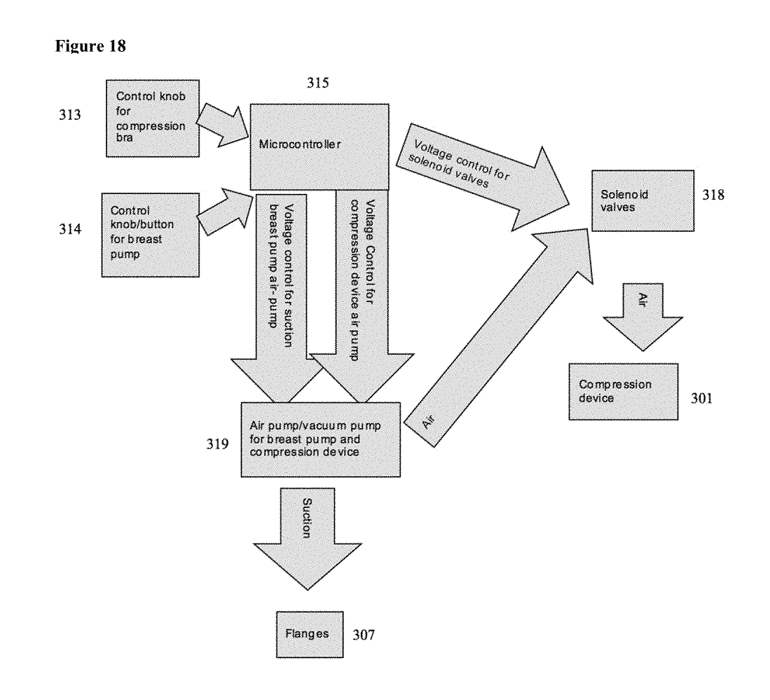

FIG. 13 illustrates a system (300) in which the compression device (301) is integrated into a vacuum-powered suction breast pump (302), and one controller (303) operates both the compression device (301) and the vacuum-powered suction breast pump (302). Each compression device (301) is composed of at least two inflatable chambers (304, 305), each chamber connected to its own air tube (306). The chambers (304, 305) inflate and deflate in an alternating pattern. Because of the design of the chambers (304, 305), the air flows from the air tube (306) into the chamber, filling a large pocket before being pushed through a smaller channel before entering another large pocket, and so on and so forth until the entire chamber has inflated in a rotational pattern. Once one chamber (304) is fully inflated, it deflates as the other chamber (305) begins to inflate. The air passes in and out of the tubes (306) connecting the compression device (301) to the controller (303). In the center of the compression device (301) is a flange (307) to connecting the compression device (301) to a vacuum-powered suction breast pump (302). The flange (307) may be connected to or embedded within the compression device (301). The flange (307) can connect to a milk storage vessel (308) and to a vacuum pump (317) (FIG. 18) housed within an embodiment of the controller (303) via a connector air tube (309). As the compression device (301) inflates and deflates, air is pulled from the area around the nipple within the flange (307) by a vacuum through a detachable tube (309). When the vacuum pump (317) is turned on and operating, and the flange (307) is positioned on a woman's breast, the flange (307) seals a volume of air to create a pressure gradient against the skin which is higher than the atmospheric pressure outside the flange. The cycling of the vacuum pump (317) simulates suction, and milk is expressed. As milk accumulates at the end of the flange (307), the milk flows into a removable milk storage vessel (308).

FIG. 14 is a perspective view of the compression device (301) as it faces the user's breast. Rigid protrusions (310) may be attached to various sections of the air bladders (304, 305) to increase the pressure onto the breast during inflation. At the center of the compression device is a flange (307) to lay on top of the breast, with the nipple in the center-most chamber (311). The flange may be constructed of any material capable of forming a vacuum-seal over the skin around the nipple and areola.

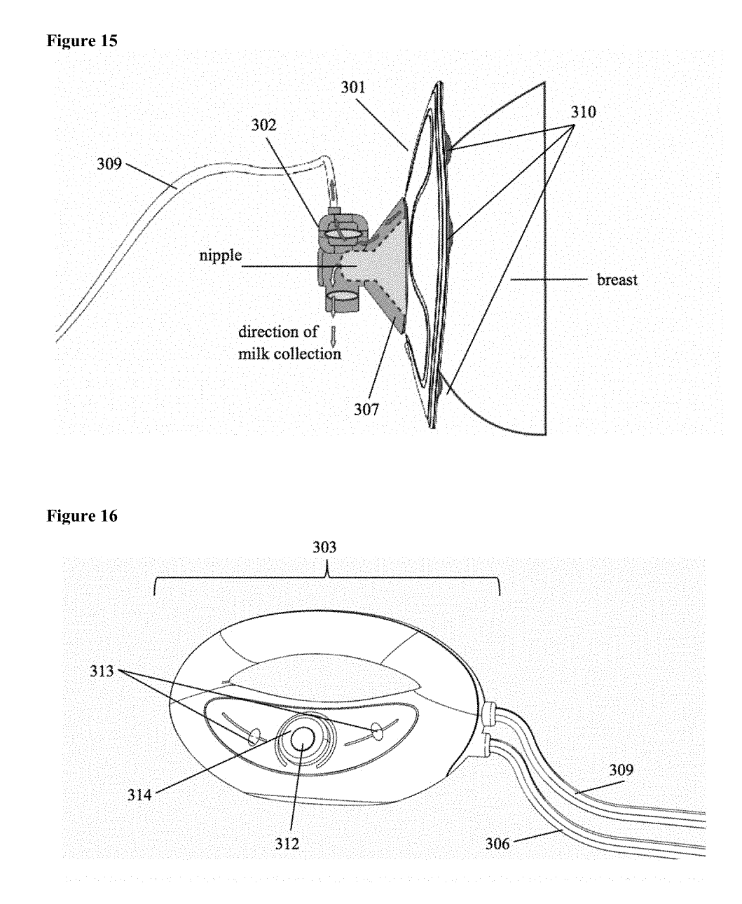

FIG. 15 is a perspective of the compression device (301) and breast pump (302) atop a user's breast. Rigid protrusions (310) face the breast so that when the bladders inflate in a rotational, hemispheric manner, the protrusions (310) apply additional pressure to the breast. FIG. 15 illustrates the placement of the nipple into the flange (307). As air is suctioned out into an air tube (309), milk is expressed and collected into a milk storage unit (308) in the direction shown.

FIG. 16 is a perspective view of a controller (303) in which a user can control the strength of compression, the strength of suction, and the pattern of compression. The user turns the device on and off by pressing the power button (312). The user can adjust the strength of compression and pattern by manipulating the controls (313), which may be represented as buttons, knobs, slide potentiometers, or digital settings. The pattern may be selected from one or more predetermined patterns or may be created by appropriate adjustment of the controls (313). The user can also adjust the strength of suction by manipulating the control knob (314) which may also be an individual or multiple buttons. Air flow is routed out through tubes (306) to the compression device (301). Air flows into the controller (303) through additional tubes (309), which can be attached to flanges (307) that when held up against the breast, create a vacuum seal against the skin that suctions the nipples in and out of the flange (307) to funnel expressed breast milk. The rhythm of suction and compression patterns can be customized through the controls (313, 314).

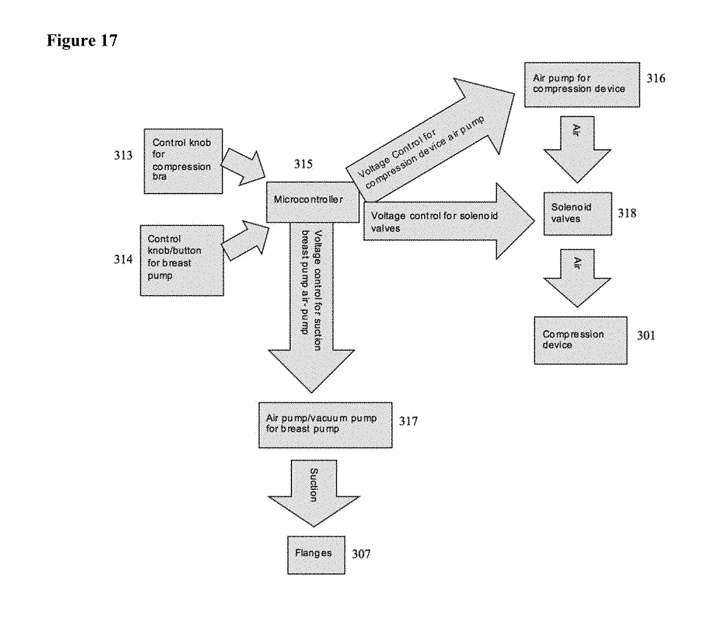

FIG. 17 is a high-level chart that displays the components of the controller (303) and how the components interact with each part of the system (300). The main components of the controller (303) include: a power switch (312), potentiometer controls (313, 314) (that may be embodied as turning knobs, sliding knobs, or buttons), a microcontroller (315), two air and suction pumps (316, 317), and solenoid air valves (318). The power switch (312) turns the device on or off by acting as a binary control for the input voltage of the entire controller (303). The voltage is provided by an AC/DC adapter or a battery (not shown). When the switch (312) is in the “on” setting, voltage is supplied to the microcontroller (315), in which the functionalities of the switch (312), the knobs and buttons (313, 314), and the control voltage for the air pumps (316, 317) and solenoid valves (318) are coded, automating the control over the entire system (300). Based on the position of the buttons and knobs (313, 314), the appropriate pattern of compression and strengths of compression and vacuum suction are read by the microcontroller (315), which controls the pumps (316, 317) by adjusting the voltage supply to them. One air and vacuum pump (316) acts as the source of compressed air for the compression device and the other (317) acts as the source of suction for the breast pump. The pumps (316, 317) contain two nozzles: one nozzle is the input suction nozzle and the other nozzle puts out compressed air. For the breast pump (302), suction creates a vacuum that is applied directly at the flanges (307), which is connected to the pump (317) via an air tube (309). For the compression device (301), the air output from the pump (316) is sent to the solenoid valves (318). The solenoid air valves are electric valves that act like a gate to the compression device (301). The solenoid valves (318) contain three nozzles: one input nozzle and two output nozzles. The air output from the air pump (316) is supplied to the input nozzle. When voltage is supplied to the solenoid valves (318), air is let out from one of the output nozzles into the air tubes (306) that lead to the compression device (301), inflating the air bladders (304, 305). When the voltage supply is cut off, the second output nozzle is open to let out the excess air of the system. The rotational hemispheric compression of the compression device (301) is due to the operation of the solenoid valves (318).

FIG. 18 is a high-level chart that displays the components of the controller (303) and how the components interact with each part of the system (300). The components are largely the same as in FIG. 17, but instead of an air and vacuum pump (316) designated to the compression device (301) and the other (317) designated to the vacuum-powered breast pump (302), there is only one air and vacuum pump (319) that services both the compression device and the breast pump. The air suctioned from the breast pump is sent to the solenoid valves (318) to be pushed into the compression device (301).

It is to be understood that while the invention has been described in conjunction with the preferred specific embodiments thereof, that the foregoing description and the examples that follow are intended to illustrate and not limit the scope of the invention. It will be understood by those skilled in the art that various changes may be made and equivalents may be substituted without departing from the scope of the invention, and further that other aspects, advantages and modifications will be apparent to those skilled in the art to which the invention pertains. In addition to the embodiments described herein, the present invention contemplates and claims those inventions resulting from the combination of features of the invention cited herein and those of the cited prior art references which complement the features of the present invention. Similarly, it will be appreciated that any described material, feature, or article may be used in combination with any other material, feature, or article, and such combinations are considered within the scope of this invention as defined by the following claims.

What is claimed is:

1. A system for expressing milk from human breasts, comprising:
a compression device configured to apply pressure in a rotational pattern around the hemispheres of the breasts to mimic manual compression, comprised of at least one inflatable bladder to lay over the breast, with an opening in the center of the device to lay over the nipple; and
a controller to control the rotational pattern applied by said compression device.

2. The system of claim 1, wherein said compression device is configured to apply compression to the breasts in a rotating, pulsing manner.

3. The system of claim 1, wherein said compression device is pneumatically actuated through tubes that pass from said controller into said compression device.

4. The system of claim 3, wherein said controller increases or decreases the pressure exerted by said compression device by increasing or decreasing the fluid pumped.

5. The system of claim 1, wherein said compression device is incorporated into a bra to be worn by the user, and said bra has in each cup an opening to lie over the nipple.

6. The system of claim 5, wherein said compression device is comprised of a plurality of inflatable arms arranged radially around the nipple.

7. The system of claim 6, wherein said arms vary in length to reach the tail of Spence, the outer diagonal hemisphere of the breast, or the inner hemispheres of the breast.

8. The system of claim 6, wherein said arms each have their own tube connecting them to the controller.

9. The system of claim 6, wherein said arms share air tubes.

10. The system of claim 6, wherein said arms inflate and deflate in rotating hemispheric massage patterns.

11. The system of claim 5, wherein said compression device is disc-shaped, with an opening in the center to lay over the nipple.

12. The system of claim 5, wherein said compression device includes inflatable bladders arranged such that inflation proceeds in a rotational manner.

13. The system of claim 12, wherein said inflatable bladders have one or more sealed chambers, each designed such that air passes from the air tube into a larger pocket, proceeding through smaller channels into other larger pockets, inflating the chambers rotationally.

14. The system of claim 12, wherein at least one small rigid protrusion is attached to said compression device and facing the user's breast for additional pressure when said bladder is inflated.

15. The system of claim 12, wherein a semi-rigid element applied to said inflatable bladders prevents said inflatable bladders from inflating away from the breasts, forcing the pressure to compress the user's breasts.

16. The system of claim 13, wherein each chamber has its own air tube.

17. The system of claim 1, wherein said compression device is incorporated into a breast pump.

18. The system of claim 17, wherein said compression device comprises the flange of a suction breast pump.

19. The system of claim 19, wherein said controller for said compression device is incorporated into the controller for said suction breast pump.
